(19) United States
(12) Patent Application Publication
(10) Pub. No.: US 2008/0260856 A1
(43) Pub. Date: Oct. 23, 2008

(54) Topical composition containing sacred water

(76) Inventor: Karin Nightingale Hasbrouck, El Segundo, CA (US)

Correspondence Address: The Halvorson Law Firm, 1757 E. Baseline Rd., Ste 130, Gilbert, AZ, 85233, US

(21) Appl. No.: 11/787,641

(22) Filed: Apr. 17, 2007

(51) Int. Cl. A61K 33/00

(52) U.S. Cl. 424/600; 424/725

(57) ABSTRACT

Topical compositions having as at least one part of their composition sacred and/or holy water. These compositions are useful for imparting a spiritual sense of well-being.

### TECHNICAL FIELD

The present invention relates to the field of topical compositions for skin. More particularly, the invention relates to topical compositions that include as an ingredient sacred or holy water.

### BACKGROUND

Water is a primordial element which underlays creation myths and stories around the world. The Egyptian Heliopolitan creation story recounts that the sun-god Atum reposed in the primordial ocean. In Assyro-Babylonian mythology, first the gods and subsequently all beings arose from the fusion of salt water and sweet water. The holy books of the Hindus explain that all the inhabitants of the earth emerged from the primordial sea. At the beginning of the Judeo-Christian story of creation, the spirit of God is described as stirring above the waters, and a few lines later, God creates a firmament in the midst of the waters to divide the waters. In the Koran are the words We have created every living thing from water.

Water divinities of various kinds appear in the mythologies of many cultures. And not surprisingly, the world abounds in sacred springs, rivers, and lakes. Even within the Judeo-Christian tradition, which generally avoids the veneration of the various phenomena of Nature, there are numerous examples of sacred springs or wells, and rivers. In most cases, the spring or river has acquired sacredness through connection with a significant or miraculous event. The water of the River Jordan is sacred because Jesus Christ was baptized in it by Saint John the Baptist. The spring at Lourdes is sacred because of its healing properties in connection with the appearance of the Virgin Mary to Bernadette. In some cases, such as the holy well at Chartres, or the Chalice Well at Glastonbury were probably already sacred in pagan times.

While sacred in their own right, sacred springs also draw attention to the sacredness of water itself, reminding the Christian, for example, that water is a symbol of grace (and as such is used for baptism). Water is also one of the four elements possessing fundamental characteristics. In many cultures, water appears as a reflection or an image of the soul. In Japan, water prefigures the purity and pliant simplicity of life. It can be both calm and animated, and the Japanese may contemplate the unruffled surface of a temple pond or make pilgrimages to waterfalls. The lotus-stream of the Buddha or Boddhisattva rises up from the waters of the soul, in the same way the spirit, illumined by knowledge, frees itself from passive existence.

In India, the sacred River Ganges embodies for Hindus the water of life. Bathing in the Ganges frees the bather from sin, the outward purification serving as symbolic support of inward purification. The source of the Ganges lies in the Himalayas, the mountains of the Gods, and descends to the plains of India as if from Heaven.

The identification of the sources of rivers, streams, springs, and wells as sacred is very ancient. Springs and wells were perceived as the dwelling place of supernatural beings, and stories and legends grew up around them. Often it was claimed that the waters healed the injured or cured the sick with the result that well or stream came to be regarded as a sacred shrine.

In some cases wells or streams were oracular. Pausanias reports that a sacred stream in front of the sanctuary of Demeter at Patras served as an infallible mode of divination using a mirror. Wells and springs inhabited by spirits with the gift of prophecy were places of pilgrimage. The Celts venerated natural springs of water for their sacred and medicinal value and many examples of holy wells are known, many of them were later Christianized through rededication to a saint. This practice of venerating sacred wells continued into the Christian era in the West, though they were now referred to as wishing wells.

Springs and wells also took the form of sacred fountains which were claimed to be the Fountain of Youth, or the Fountain of Immortality, or the Well of Knowledge. A Fountain of Youth was believed to exist in the newly-discovered Americas, and the Spanish conquistador Ponce de Léon set out in 1513 on an expedition to find it in Florida. In China, the water of the fountain at Pon Lai was believed to confer a thousand lives on those who drink it, according to Wang Chia, writing in the Chin Dynasty, and a similar reputation was attached to the springs of Mount Lao Shan.

Wells and springs were often associated with a god or goddess and the sacred water dispensed there could ensure life, health, and abundance. The Babylonian moon goddess, Ishtar, was associated with sacred springs, and her temples were often situated in natural grottoes from which springs emanated. Sacred springs were enshrined by the Ancient Greeks who erected artificial basins and placed icons of the deity or deities nearby. Goddesses and nymphs were connected with certain rivers, springs, and wells by the Celts and Romans. Often the river was named after the goddess, such as the Shannon River, after Sinann, and the Boyne, after Boann, in Ireland, and the Seine, after Sequana, in Gaul (France). In 1963, at the Gallo-Roman Fontes Sequanae sanctuary at the source of the Seine, 200 wooden figures were exacavated carved from the heart wood of oak to represent all or part of the human body (heads, limbs, trunks; with internal organs carved in relief on wooden plaques). These ex votos indicate that the goddess of the sacred spring was believed capable of curing a whole range of infirmities.

A special sacred significance was attached to springs and wells whose waters could heal. In the New Testament, St. John (5:2) describes the pool of Bethesda in Jerusalem, surrounded by five covered colonnades, where a great number of disabled people used to lie—the blind, the lame, the paralyzed waiting to be the first to enter the pool when the water is stirred. When in the mid-19th century soon after Bernadette's vision of the Virgin Mary, the water issuing from the grotto at Lourdes began to bring about cures in people, the spring was designated a place of miracles.

It is evident that people take great comfort and derive great benefit from the properties of sacred, and/or holy water. Thus, there is a current and ongoing need for products that include sacred and/or holy, water as a component in their compositions. As used herein, Holy Water is water that has been blessed by spiritual leaders.

Whilst the compositions and disclosures of the prior art provide useful advances in the art of cosmetic skin treatment, they do not include the spiritual and healing properties of scared and/or holy water.

### SUMMARY OF THE INVENTION

The present invention relates to topical compositions having as at least one part of their composition sacred and/or holy water.

These compositions are useful for imparting a spiritual sense of well-being.

### DETAILED DESCRIPTION OF THE INVENTION

All percentages and ratios used herein are by weight of the total composition and all measurements made are at 25° C., unless otherwise designated.

All publications cited herein are hereby incorporated by reference in their entirety.

The term “dermatologically-acceptable,” as used herein, means that the compositions or components thereof so described are suitable for use in contact with human skin without undue toxicity, incompatibility, instability, allergic response, and the like.

The term “safe and effective amount” as used herein means an amount of a compound, component, or composition sufficient to significantly induce a positive benefit, preferably a positive, spiritual feeling, skin appearance or feel benefit, including independently the benefits disclosed herein, but low enough to avoid serious side effects, i.e., to provide a reasonable benefit to risk ratio, within the scope of sound medical judgment.

Active and other ingredients useful herein may be categorized or described herein by their spiritual, cosmetic and/or therapeutic benefit or their postulated mode of action. However, it is to be understood that the active and other ingredients useful herein can in some instances provide more than one cosmetic and/or therapeutic and/or spiritual benefit or operate via more than one mode of action. Therefore, classifications herein are made for the sake of convenience and are not intended to limit an ingredient to the particularly stated application or applications listed.

The compositions of the invention are useful for topical application and for providing an essentially immediate improvement in skin feel and appearance following application of the composition to the skin. The compositions of the invention are also useful for providing improvements in skin appearance or condition following multiple topical applications of the composition to the skin. The compositions provide the benefits along with a spiritual benefit.

More particularly, the compositions of the present invention are useful for imparting a sense of spiritual well being along with the regulation of skin conditions.

The present novel compositions are formulated into an aqueous base system comprising sacred or holy water.

The aqueous base system of the present invention provides an additional improvement over currently available compositions because the system allows for the spiritual well being of a user.

This invention constitutes the first use, known to the inventors, of sacred or holy water in these types of products, i.e., a self-action tanning product, tan extending lotions sunless tanning spray, exfoliating washes, cosmetic preparations, toiletries and the like.

Suitable sacred or holy water sources for use in the present invention are Lourdes spring water, water from sacred springs, wells or rivers, water blessed by the Catholic or other spiritual leaders and the like.

Various optional ingredients may be included in the compositions of the present invention, these include but are not limited to perfumes, preservatives, emollients, antiseptics, pigments, dyes, humectants, propellants, as well as other classes of materials whose presence may be cosmetically, medicinally or otherwise desirable. Common examples can be found in the CTFA International Cosmetic Ingredient Dictionary 4th Edition, The Cosmetic, Toiletry, and Fragrance Association, Inc., Washington, D.C., 1991. Common examples of such ingredients are provided below by way of example and not limitation.

Optional ingredients include polyoxyethylene ethers such as PPG-12-buteth-16 (UCON 50 HB 660), PPG-3-buteth 5, PPG-5-buteth 7, PPG-7-buteth 10, PPG-9-buteth 12, PPG-12-buteth 16, PPG-15-buteth 20, PPG-20-buteth 30, PPG-28-buteth 35, PPG-33-buteth 45, PEG-4, PEG-6, PEG-8, PEG-10, PEG-12, PEG-32, or suitable ingredients which provide emolliency; hydrolyzed wheat protein/wheat oligosaccharides such as Cropeptide W.®, hydrolyzed corn protein, hydrolyzed corn starch, hydrolyzed wheat gluten, hydrolyzed yeast protein, hydrolyzed vegetable protein, hydrolyzed soy protein, hydrolyzed rice protein, hydrolyzed potato protein, which are suitable for moisturization; polyethylene glycol esters such as PEG-14 Laurate, PEG-15 Laurate, PEG-20 Laurate, PEG-32 Laurate, PEG-75 Laurate, PEG-150 Laurate or other surfactants which are used for fragrance solubilization; glycereth-7-triacetate (Dermol GL-7A, Alzo), glycerin, glycereth 5 lactate, glycereth 7 diisonanoate which are used for moisturization, emolliency and to help solubilize fragrance; PEG-40 Castor Oil (Surfactol 365, CasChem), PEG 45 castor oil, PEG 50 castor oil, PEG 60 castor oil, PEG 100 castor oil which are surfactants to help solubilize fragrance and also provide emolliency and moisturization; preservatives such as methyldibromo-glutaronitrile/phenoxyethanol/polyquaternium-7 (Euxyl K-400, Calgon) methyl paraben, imidazolidinyl urea benzalkonium chloride, diazolidinyl urea, benzethonium chloride, sodium benzoate and sorbic acid; sunscreens such as octyldimethyl PABA, benzophenone-4, DEA-methoxycinnamate, 2-phenyl-benzimidazole-5-sulphonic acid and TEA salicylate; and finally fragrances. As used herein, the term “alcohols” refers to common solvents known to those skilled in the cosmetic art; these include but are not limited to ethanol and isopropyl alcohol.

The compositions of the present invention are prepared by the following preferred procedure that is performed at room temperature and pressure. Other procedures known to those skilled in the art may also be used.

The compositions of the present invention are generally prepared by conventional methods such as are known in the art of making topical compositions. Such methods typically involve mixing of the ingredients in one or more steps to a relatively uniform state, with or without heating, cooling, application of vacuum, and the like.

A wide range of quantities of the compositions of the present invention can be employed to provide sense of spiritual well being and/or a skin appearance and/or feel benefit. Typically applications would be on the order of about once per day, however application rates can vary from about once per week up to about three times per day or more. Also, these compositions may be used on a monthly, seasonal or other basis.

In addition to the sense of spiritual well being, the compositions of this invention provide an improvement in skin condition following application of the composition to the skin.

Application of the sacred or holy water, by itself is considered to provide a feeling of spiritual well being in individuals.

### EXAMPLES

The following examples further describe and demonstrate embodiments within the scope of the present invention. The examples are given solely for the purpose of illustration and are not to be construed as limitations of the present invention. Where applicable, ingredients are given in CTFA name.

### Example 1 Sunless Tanning Spray

| | Preferred % of | | |
|---|---|---|---|
| Ingredient | Composition | Lower Range | Upper Range |
| Deionized Purified | 53.30 | 0.00 | 54.29 |
| Water | | | |
| Sacred Water or | 1.00 | 0.01 | 54.30 |
| Holy Water | | | |
| Dihydroxyacetone | 6.00 | 2.00 | 10.00 |
| Citric Acid | 0.10 | 0.00 | 1.0 |
| Aloe Vera Extract | 15.00 | 0.00 | 30.00 |
| PEG 16 Macadamia | 2.00 | 0.00 | 4.00 |
| Glycerides | | | |
| Ethoxydiglycol | 3.00 | 0.00 | 6.00 |
| SD40 B Denatured | 3.00 | 0.00 | 6.00 |
| Alcohol | | | |
| Vitamin E Acetate | 0.25 | 0.00 | 0.50 |
| Polysorate 20 | 2.00 | 0.00 | 4.00 |
| Propylene Glycol | 10.00 | 0.00 | 20.00 |
| Fragrance | 0.25 | 0.00 | 0.50 |
| Vitamin C | 0.10 | 0.00 | 0.20 |
| Papaya Extract | 1.00 | 0.00 | 2.00 |
| Green ea Extract | 1.00 | 0.00 | 2.00 |
| Awapuhi Extract | 1.00 | 0.00 | 2.00 |
| Cucumber Extract | 1.00 | 0.00 | 2.00 |

### Example 2 Tan Extending Lotion

| | Preferred % of | | |
|---|---|---|---|
| Ingredient | Composition | Lower Range | Upper Range |
| Deionized Purified | 43.95 | 0.00 | 44.94 |
| Water | | | |
| Sacred or Holy Water | 1.00 | 0.01 | 44.95 |
| Aloe Vera Gel | 20.00 | 0.00 | 40.00 |
| Behenyl Alcohol | 3.00 | 0.00 | 6.00 |
| Isopentydiol | 3.00 | 0.00 | 6.00 |
| Safflour Oil | 2.00 | 0.00 | 4.00 |
| Squalane | 2.00 | 0.00 | 4.00 |
| Capric Capriylic | 2.50 | 0.00 | 5.00 |
| Triglycerides | | | |
| Glyceryl Stearate | 5.00 | 0.00 | 1.00 |
| Peg-100 Stearate | 2.50 | 0.00 | 5.00 |
| Papaya Extract | 1.00 | 0.00 | 2.00 |
| Green Tea Extract | 1.00 | 0.00 | 2.00 |
| Awapuhi Extract | 1.00 | 0.00 | 2.00 |
| Cucumber Extract | 1.00 | 0.00 | 2.00 |
| Shea Butter | 2.00 | 0.00 | 4.00 |
| Cocoa Butter | 2.00 | 0.00 | 4.00 |
| Avocado Oil | 2.00 | 0.00 | 4.00 |
| Kukui Nut Oil | 1.00 | 0.00 | 2.00 |
| Hydroexythylcellulose | 0.25 | 0.00 | 0.50 |
| Dihydroxyacetone | 1.00 | 0.01 | 2.00 |
| Disodium EDTA | 0.10 | 0.00 | 0.20 |
| Tocopherol Acetate | 1.00 | 0.00 | 2.00 |
| Sodium Hydroxide | 0.10 | 0.00 | 0.20 |
| Vitamin B5 | 0.10 | 0.00 | 0.20 |
| Phenoxyethanol | 0.65 | 0.00 | 1.30 |
| Ethylhexylglycerine | 0.10 | 0.00 | 0.20 |
| Hexylene Glycol | 0.10 | 0.00 | 0.20 |
| Capryl Glycol | 0.15 | 0.00 | 0.30 |
| Fragrance | 0.50 | 0.01 | 1.00 |

### Example 3 Fixed Fruit Acid Exfoliating Wash

| | Preferred % of | | |
|---|---|---|---|
| Ingredient | Composition | Lower Range | Upper Range |
| Deionized Purified | 66.50 | 0.00 | 67.49 |
| Water | | | |
| Sacred or Holy | 1.00 | 0.01 | 67.50 |
| Water | | | |
| Papaya Extract | 1.00 | 0.00 | 2.00 |
| Green Tea Extract | 1.00 | 0.00 | 2.00 |
| Awapuhi Extract | 1.00 | 0.00 | 2.00 |
| Cucumber Extract | 1.00 | 0.00 | 2.00 |
| Sodium Laureth | 10.00 | 0.00 | 20.00 |
| Sulfate | | | |
| Cocamidopropyl | 3.00 | 0.00 | 6.00 |
| Betaine | | | |
| Cocamide DEA | 3.00 | 0.00 | 6.00 |
| Aloe Vera Juice | 5.00 | 0.00 | 10.00 |
| Cocomidopropyl | 2.00 | 0.00 | 4.00 |
| Hydroxysultaine | | | |
| Tocopherol Acetate | 0.25 | 0.00 | 0.50 |
| DMDM Hydaintion | 0.30 | 0.00 | 0.60 |
| Natural Sea Salt | 1.00 | 0.00 | 2.00 |
| Fragrance | 0.50 | 0.00 | 1.00 |
| FD&C Blue #1 | 0.00010 | 0.00 | 0.00020 |
| Polysorbate 20 | 2.00 | 0.00 | 4.00 |
| Sugar cane Extract | 1.00 | 0.00 | 2.00 |
| Citric Acid | 0.20 | 0.00 | 0.40 |
| Lactic Acid | 0.25 | 0.00 | 0.50 |

### Example 4 Sunless Tanning Lotion

| | Preferred % of | | |
|---|---|---|---|
| Ingredient | Composition | Lower Range | Upper Range |
| Deionized Purified | 39.80 | 0.00 | 40.79 |
| Water | | | |
| Sacred or Holy Water | 1.00 | 0.10 | 40.80 |
| Aloe Vera Gel | 20.00 | 0.00 | 40.00 |
| Behenyl Alcohol | 3.00 | 0.00 | 6.00 |
| Isopentydiol | 3.00 | 0.00 | 6.00 |
| Safflour Oil | 2.00 | 0.00 | 4.00 |
| Squalane | 2.00 | 0.00 | 4.00 |
| Capric Caprylic | 2.50 | 0.00 | 5.00 |
| Triglycerides | | | |
| Glyceryl Stearate | 5.00 | 0.00 | 10.00 |
| PEG-100 Stearate | 2.50 | 0.00 | 5.00 |
| Papaya Extract | 1.00 | 0.00 | 2.00 |
| Green Tea Extract | 1.00 | 0.00 | 2.00 |
| Awapuhi Extract | 1.00 | 0.00 | 2.00 |
| Cucumber Extract | 1.00 | 0.00 | 2.00 |
| Shea Butter | 2.00 | 0.00 | 4.00 |
| Cocoa Butter | 2.00 | 0.00 | 4.00 |
| Avacado Oil | 2.00 | 0.00 | 4.00 |
| Kukui Nut Oil | 1.00 | 0.00 | 2.00 |
| Hydroxyethylcellulose | 0.25 | 0.00 | 0.50 |
| Dihydroxyacetone | 5.00 | 0.10 | 10.00 |
| Disodium EDTA | 0.10 | 0.00 | 0.20 |
| Tocopherol Acetate | 1.00 | 0.00 | 2.00 |
| Sodium Hydroxide | 0.10 | 0.00 | 0.20 |
| Vitamin B5 | 0.10 | 0.00 | 0.20 |
| Phenoxyethanol | 0.65 | 0.00 | 1.30 |
| Ethylhexylglycerine | 0.10 | 0.00 | 0.20 |
| Hexylene Glycol | 0.10 | 0.00 | 0.20 |
| Capryl Glycol | 0.15 | 0.00 | 0.30 |
| Citric Acid | 0.15 | 0.00 | 0.30 |
| Fragrance | 0.50 | 0.00 | 1.00 |

The preferred embodiment of the invention is described above in the Drawings and Description of Preferred Embodiments. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s). The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated.

What is claimed is:

1. A topical composition comprising an effective amount of sacred or holy water and non-holy water.

2. The composition according to claim 1 wherein the non-holy water is deionized water.

3. The composition according to claim 1 wherein the composition further comprises Dihydroxyacetone.

4. The composition according to claim 1 wherein the composition further comprises an antioxidant.

5. The composition according to claim 1 wherein the composition further comprises an emollient.

6. The composition according to claim 1 wherein the composition further comprises a surfactant.

7. The composition according to claim 1 wherein the composition further comprises natural products.

8. The composition according to claim 1 wherein the composition further comprises particulates.

9. A method for generating spiritual well being in an individual comprising the steps of applying a topical composition to the skin of the individual, said topical composition comprising an effective amount of sacred or holy water and non-holy water.
The composition according to claim 1 wherein the non-holy water is deionized water.

10. The method according to claim 9 wherein the composition further comprises Dihydroxyacetone.

11. The method according to claim 9 wherein the composition further comprises an antioxidant.

12. The method according to claim 9 wherein the composition further comprises an emollient.

13. The method according to claim 9 wherein the composition further comprises a surfactant.

14. The method according to claim 9 wherein the composition further comprises natural products.

15. The method according to claim 9 wherein the composition further comprises particulates.

16. The method according to claim 9 wherein the non-holy water is deionized water.
